(19) United States
(12) Patent Application Publication
(10) Pub. No.: US 2006/0130829 A1
(43) Pub. Date: Jun. 22, 2006

(54) Inhalation device

(76) Inventors: Douglas A. Sexton, La Jolla, CA (US); Winthrop D. Childers, San Diego, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/017,163

(22) Filed: Dec. 20, 2004

(51) Int. Cl. A61M 11/00; G06F 19/00

(52) U.S. Cl. 128/200.23; 705/3

(57) ABSTRACT

An inhalation device includes an ejector head having one or more drop generator(s). A reservoir, adapted to contain a pharmaceutically active ingredient therein, is in selective fluid communication with the drop generator(s). Electronic circuitry is in electronic communication with, and operatively controls the drop generator(s). Further, the electronic circuitry is responsive to either a predetermined fault condition or an operational condition. The electronic circuitry deactivates the drop generator(s) in response to the predetermined fault condition, and the electronic circuitry activates the drop generator(s) in response to the operational condition.

### BACKGROUND

The present disclosure relates generally to inhalation devices.

Pharmaceutically active ingredients may include various drugs that exhibit opium or morphine-like properties, such as, for example opioids. Opioids are often administered to patients as analgesics, but have many other pharmacological effects, including drowsiness, respiratory depression, mood swings, and mental clouding without loss of consciousness. Opioids act as agonists as they interact with stereospecific and saturable binding sites in the brain and other tissues. Endogenous opioid-like peptides may be present in areas of the central nervous system that may be related to pain perception, movement, mood, behavior, and the regulation of neuroendocrinological functions. Opium contains more than twenty distinct alkaloids, including morphine, codeine and papaverine.

Repeated opioid use may lead to the development of tolerance, physical dependence, and/or psychological dependence (i.e., addiction) thereon. A concern in using opioids for the treatment of pain is the potential development of such tolerance and/or addiction. Another major concern is the transportation of these drugs from the patient to a non-patient for recreational purposes.

There have previously been attempts to control the potential abuse of opioids. Particular doses of opioids may be more potent when administered parenterally than when administered orally. Attempts to reduce or prevent abuse have included adding an antagonist to the oral dosage form which is not orally active but which will substantially block the analgesic/euphoric effects of the opioid if an attempt is made to dissolve the opioid and administer it parenterally.

Attempts have also been made to control the potential abuse of opioids contained within inhalation systems. These attempts include some form of “lock and key” to allow a certain patient access to the opioid. However, the potential of abuse may remain, as the “keys” could be shared with others, or the device could be tampered with in an attempt to remove and potentially abuse the opioid.

As such, it would be desirable to provide an inhalation system that substantially prevents abuse of a pharmaceutically active ingredient contained therein.

### SUMMARY

An inhalation device is disclosed. The inhalation device includes an ejector head having one or more drop generator(s). A reservoir, adapted to contain a pharmaceutically active ingredient therein, is in selective fluid communication with the drop generator(s). Electronic circuitry is in electronic communication with, and operatively controls the drop generator(s). Further, the electronic circuitry is responsive to either a predetermined fault condition or an operational condition. The electronic circuitry deactivates the drop generator(s) in response to the predetermined fault condition, and the electronic circuitry activates the drop generator(s) in response to the operational condition.

### BRIEF DESCRIPTION OF THE DRAWINGS

Objects, features and advantages will become apparent by reference to the following detailed description and drawings, in which like reference numerals correspond to similar, though not necessarily identical components. For the sake of brevity, reference numerals having a previously described function may not necessarily be described in connection with subsequent drawings in which they appear.

FIG. 1 is a flow diagram depicting an embodiment of a method of making a pharmaceutically active ingredient abuse-prevention device;

FIG. 2 is a schematic view of an embodiment of a system for preventing abuse of a pharmaceutically active ingredient;

FIG. 3 is a flow diagram depicting an embodiment of a method as disclosed herein;

FIG. 4 is a flow diagram depicting an alternate embodiment of a method as disclosed herein;

FIG. 5 is a perspective semi-schematic cutaway view of an embodiment of an inhaler having an embodiment of a drop generator therein;

FIG. 6 is a semi-schematic view of an alternate embodiment of an inhaler;

FIG. 7 is a schematic perspective view of an embodiment of an ejector head; and

FIG. 8 is an enlarged, cross-sectional semi-schematic view taken along line 8-8 of FIG. 7.

### DETAILED DESCRIPTION

Embodiments of the present disclosure advantageously provide a method for preventing abuse of a pharmaceutically active ingredient (non-limitative examples of which include medications/medicants, opioids, combinations thereof, and/or the like). The method generally includes providing a pharmaceutically active ingredient in fluid communication with a drop generator. The drop generator may be advantageously electronically controlled such that, upon exposure to and/or recognition of certain fault conditions or requests, the drop generator is deactivated. As such, electronic controls (a non-limitative example of which includes drive circuitry) allow the drop generator to be rendered substantially disabled when, for example, the pharmaceutically active ingredient has expired, an unauthorized user attempts to use the pharmaceutically active ingredient, and/or someone attempts to abuse the pharmaceutically active ingredient. It is to be understood that when the drop generator is disabled, the pharmaceutically active ingredient is no longer releasable from the device/system in which it is contained.

In an alternate embodiment of the method, the drop generator is electronically controlled such that, upon exposure to an operational condition, the drop generator is activated. Prior to the activation, the user may not access the pharmaceutically active ingredient contained within the device/system.

A system for preventing pharmaceutically active ingredient abuse and an inhaler incorporating the various embodiments of the system are also disclosed herein.

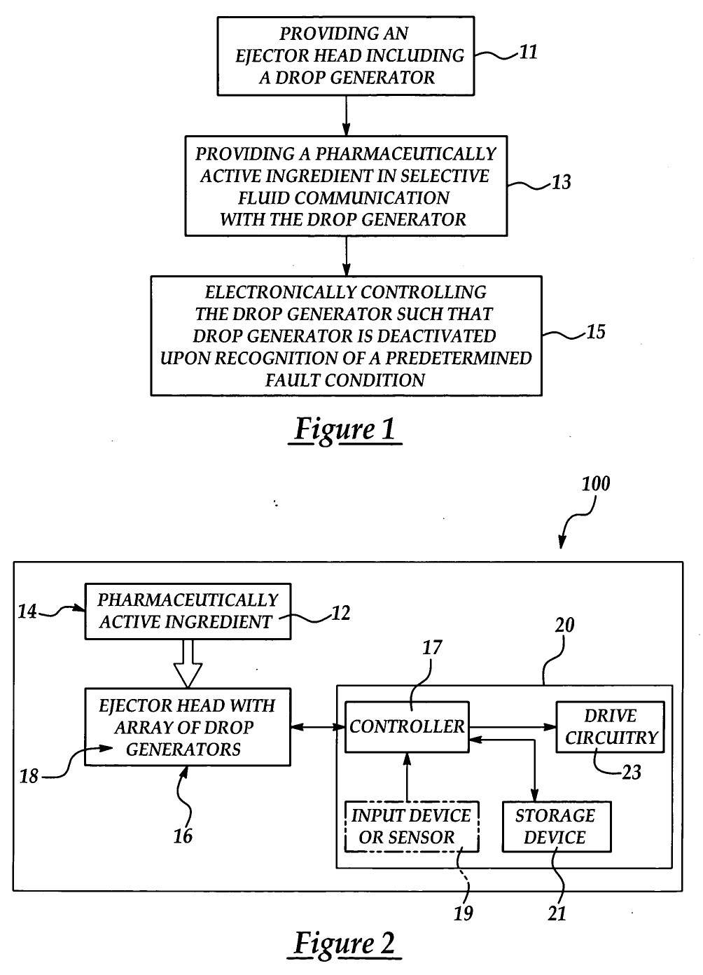

Referring now to FIG. 1, a flow diagram of an embodiment of a method of making a pharmaceutically active ingredient abuse-prevention device is depicted. An ejector head having a drop generator is provided, as shown at reference numeral 11. A pharmaceutically active ingredient is placed in fluid communication with the drop generator, as shown at reference numeral 13. An embodiment of the method may also include disposing the pharmaceutically active ingredient in a reservoir. The drop generator is electronically, operatively controlled (e.g. via electronic circuitry) so that, upon recognition of a predetermined fault condition, the drop generator is deactivated, as shown at reference numeral 15. It is to be understood that embodiment(s) of the method will be referred to in more detail in reference to FIGS. 2 through 7.

Referring now to FIG. 2, a general embodiment of a system 100 for preventing abuse of a pharmaceutically active ingredient is depicted. It is to be understood that the large arrow generally represents fluid/selective fluid pathways, and that the small arrows generally represent electronic pathways.

As depicted, a reservoir 14 is adapted to contain a pharmaceutically active ingredient 12. An embodiment of an ejector head 16 has a drop generator 18, or an array of drop generators 18, which is in operative and selective fluid communication with/selectively fluidly coupled to the reservoir 14 and the pharmaceutically active ingredient 12 when contained therein.

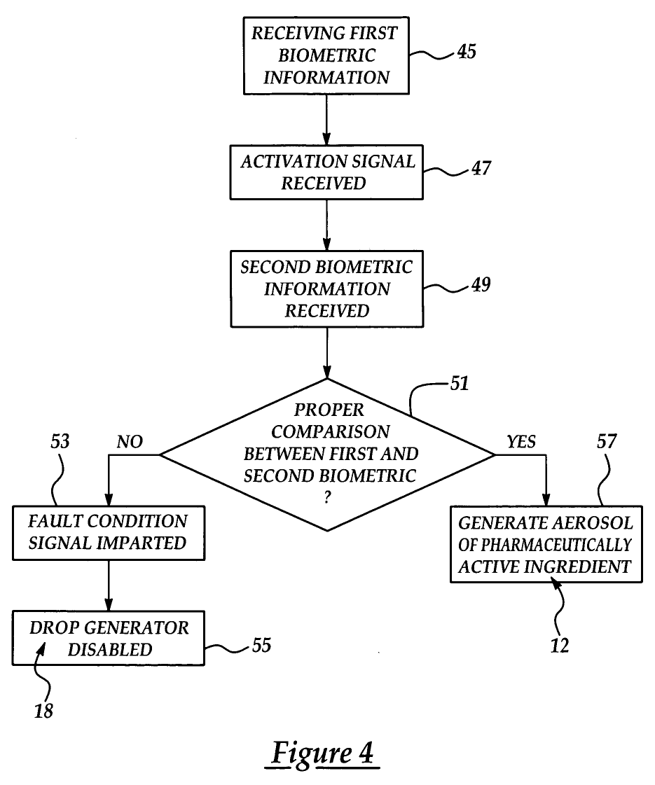

It is to be understood that any suitable pharmaceutically active ingredient 12 may be used in embodiments of the system 100, inhaler/inhalation device 10 (as shown in FIG. 4), and methods disclosed herein.

The pharmaceutically active ingredient 12 may include those substances having the capacity to produce one or more of the following: a physical dependence in which withdrawal causes sufficient distress to bring about drug-seeking behavior; the ability to assuage withdrawal symptoms caused by withdrawal from other drugs; euphoria; and patterns of toxicity resulting from a dosage above a normal therapeutic range.

In a non-limitative embodiment, the pharmaceutically active ingredient is an opioid. The term “opioid” includes stereoisomers thereof, metabolites thereof, salts thereof, ethers thereof, esters thereof, derivatives thereof, and/or mixtures thereof. Non-limitative examples of opioids include anileridine, allylprodine, alfentanil, alphaprodine, benzylmorphine, buprenorphine, bezitramide, butorphanol, codeine, clonitazene, cyclazocine, dezocine, desomorphine, dihydromorphine, dextromoramide, diampromide, dihydrocodeine, diethylthiambutene, dimenoxadol, dimepheptanol, dimethylthiambutene, dipipanone, dioxaphetyl butyrate, eptazocine, ethylmorphine, ethylmethylthiambutene, etonitazine, ethoheptazine, fentanyl, hydrocodone, heroin, 6-hydroxymorphone, hydroxypethidine, hydromorphone, isomethadone, ketobemidone, levallorphan, levophenacylmorphan, lofentanil, levorphanol, morphine, myrophine, meperidine, meptazinol, metazocine, methadone, metopon, narceine, nalbuphine, nalorphine, nicomorphine, norlevorphanol, normethadone, normorphine, norpipanone, opium, oxycodone, oxymorphone, piritramide, papaveretum, pentazocine, phenadoxone, phenazocine, phenoperidine, piminodine, phenomorphan, propheptazine, promedol, properidine, propiram, propoxyphene, sufentanil, tilidine, tramadol, stereoisomers thereof, metabolites thereof, salts thereof, ethers thereof, esters thereof, and/or derivatives thereof, and/or mixtures thereof.

It is to be understood, however, that the pharmaceutically active ingredient 12 may be any controlled substance. Non-limitative examples of such substances include testosterone and/or anabolic steroids. While such substances generally do not have euphoric effects, they may be diverted in mass. Still further, the pharmaceutically active ingredient 12 may be sedatives and/or anti-anxiety medications, as well as any combination of any of the above substances.

Electronic/control circuitry 20 may control the selective fluid communication/coupling between the reservoir 14 and the drop generator(s) 18. Further, the electronic circuitry 20 may be in electronic communication with, and may operatively control the drop generator(s) 18. In an example embodiment, electronic circuitry 20 includes a controller 17, an input or sensing device 19, a storage device 21 (e.g. a device capable of storing patient and/or other information), and/or drive circuitry 23. The controller 17 is configured to receive input from the input or sensing device 19; receive signals from, and send control signals to the ejector head 16 (described in reference to FIG. 6); exchange information with the storage device 21; and/or provide control signals to the drive circuitry 23, which activates or deactivates the drop generator(s) 18.

It is to be understood that the input or sensing device 19 may be partially or substantially wholly incorporated into the electronic circuitry 20. The input or sensing device 19 is configured to impart a “fault” or “end state” condition signal to the controller 17 in the event that, for example, expiration, abuse, and/or exhaustion of the pharmaceutically active ingredient 12 occurs. It is to be understood that a “fault condition” may also be imparted within any portion of the electronic circuitry 20 or outside of the electronic circuitry 20. The input or sensing device 19 may include any or all of the following:

(a) A sensor configured to sense tampering of the inhaler 10 or system 100, such as an attempt to access the active ingredient 12. It is to be understood that the sensor may generate a signal that is passed to the controller 17 in the event of tampering.

(b) A sensor configured to sense the opening of an access door (not shown) in the inhaler 10 or system 100.

(c) A timer system configured to generate an expiration or fault signal upon reaching a certain time limit for use of the inhaler 10, system 100, and/or the active ingredient 12.

(d) A fluid level or volume indication system configured to provide an indication of an empty condition or fault condition when it is estimated or determined that the active ingredient 12 is no longer sufficient to allow proper operation of the inhaler 10 or system 100.

(e) A system for determining malfunction of one or more portions of the inhaler 10 or system 100.

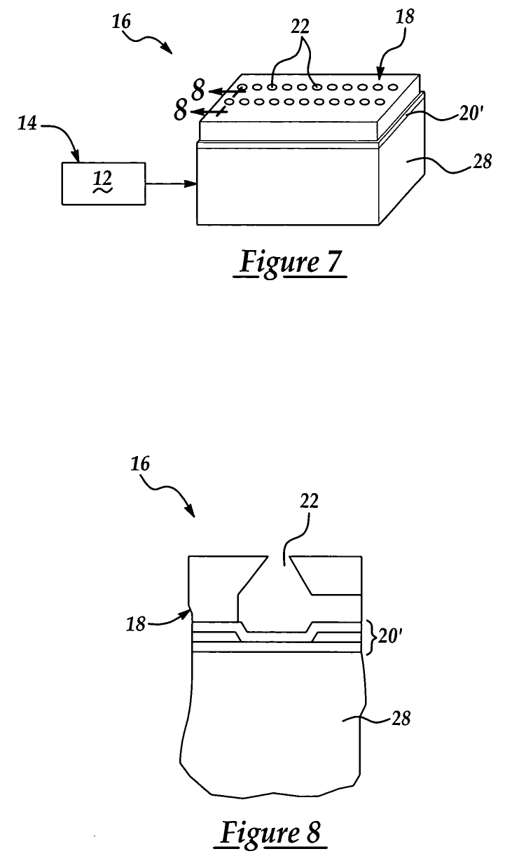

In an embodiment, the controller 17 provides control signals to the ejector head 16 for control of any drop ejection elements in the ejector head 16. In an example embodiment, the ejector head 16 includes drop generator circuitry 20′ (shown in FIGS. 6 and 7) that receives data, power, gate activation, fire-pulse, and/or other signals from the controller 17 for driving the drop generators 18 in the ejector head 16 depending, in part, on the specific electronic configuration of the ejector head 16. The controller 17 also receives signals from the ejector head 16 indicative of a state or condition of the ejector head 16, such as, for example, a temperature of a portion of the ejector head 16.

The storage device 21 may store information pertaining to the inhaler 10 (or system 100), the patient, and/or the pharmaceutically active ingredient 12. Non-limitative examples of such information include information indicative of an initial state of the inhaler 10 or system 100, a current state of the inhaler 10 or system 100, an amount of active ingredient 12 initially or remaining in the reservoir 14, an expiration date of the ingredient 12, an identity of the patient, an identity (e.g. serial number) of the reservoir 14, whether a fault condition has occurred, and the like, and combinations thereof. In a non-limitative example, the storage device 21 is a non-volatile memory device (NVM device). In other embodiments, the storage device 21 may include fusible links or other means for storing information.

It is to be understood that if a fault condition is imparted to the controller 17, the controller 17 applies signals to the drive circuitry 23 that in turn applies power signals to deactivate the drop generator(s) 18.

Non-limitative examples of suitable predetermined fault conditions that the electronic circuitry 20 may recognize include the following: system 100 or inhaler 10 tampering (non-limitative examples of which include disassembly of the reservoir 14 and drilling into the reservoir 14), pharmaceutically active ingredient 12 expiration, pharmaceutically active ingredient 12 overuse or misuse, attempted re-use after system 100 or inhaler 10 disposal, unauthorized use, loss of reservoir 14 back pressure, reuse of a single use reservoir 14, reservoir 14 leaking, loss of control circuitry 20 power, user request (e.g. inputting an electronic code which signals the controller 17), and combinations thereof.

In response to receiving the one or more of the predetermined fault conditions, the electronic circuitry 20 (e.g. via the drive circuitry 23) deactivates or disables the drop generator(s) 18 such that the pharmaceutically active ingredient 12 may no longer be dispensed from the system 100 or inhaler 10. In a non-limitative example, upon recognizing one or more predetermined fault condition(s), the control circuitry 20 imparts an electrical surge to the drop generator(s) 18, thereby rendering the drop generator(s) 18 substantially permanently deactivated. It is to be understood upon drop generator 18 deactivation, a user is no longer able to release the pharmaceutically active ingredient 12 from the system 100 or inhaler 10, which may advantageously substantially prevent abuse of the ingredient 12.

In an alternate embodiment, the sensing device 19 is capable of recognizing one or more operational condition(s), and the storage device 21 (non-limitative examples of which include a write-once memory device or NVM device) is capable of storing enabling information or operating parameter(s) therein. In this embodiment, the electronic circuitry 20 is capable of comparing the enabling information to the operational condition(s). If the electronic circuitry 20 recognizes that the operational conditions fit into predetermined limits based on the enabling information, the drop generator(s) 18 may be activated, and the system 100 or inhaler 10 may be used. As defined herein, operational conditions “substantially matching” enabling information is intended to mean that the operational conditions fit into predetermined limits based on the enabling information.

It is to be understood that, generally, a medical care provider or medical professional inputs the enabling information into the storage device 21 prior to prescribing the system 100 or inhaler 10 to the user. The enabling information may include one or more of the following: a key code, a date code, reservoir fluid capacity, biometric input, and/or combinations thereof.

A key code may be a set of numbers, letters, or combinations thereof that are unique to the system 100 or inhaler 10. In an embodiment, the key code may correlate with a serial number, for example, a manufacturer's serial number. In a non-limitative example, a pharmacist may enter the key code into the storage device 21 of the inhaler 10 upon receiving a prescription for a particular user. Without entering the same key code, the user may not activate the inhaler 10. It is to be understood that the inhaler 10 or system 100 may include a keypad or an RF tag (discussed hereinbelow), with which the user may enter the key code.

A date code may be an expiration date for the system 100 or inhaler 10. In this non-limitative example, each time a user attempts to utilize the inhaler 10, a comparison is made between the current date and the stored expiration date. If the current date predates the stored expiration date, the drop generator 18 may be activated.

The initial fluid capacity of the reservoir 14 may be stored in the storage device 21 as enabling information. In a non-limitative example, the current amount of fluid in reservoir 14, upon a user's attempt to activate the system 100 or inhaler 10, may be an operational condition. For example, if the current fluid amount falls to a predetermined level below the initial fluid capacity, the drop generator will not be activated.

An example of a biometric input is a set of parameters characterizing a first graphical or spline representation of the user's fingerprint. The first graphical or spline representation may be stored in the storage device 21. In a non-limitative example, input sensor 19 may include a finger print detection chip 19. The finger print detection chip 19 outputs a signal indicative of a second graphical or spline representation of a finger contacting the finger print detection chip 19. A comparison is made between the first graphical or spline representation with the signal generated by the finger print detection chip 19 each time the inhaler 10 or system 100 actuation is attempted. If the comparison meets certain criteria, then the controller 17 allows activation of the drop generators 18.

A radio frequency (RF) Tag held by the patient can be utilized in a manner that is similar to the biometric input. The RF tag would be used to provide a signal to the controller 17 that is indicative of a key code. The key code from the RF tag would be compared with a key code initially stored on the storage device 21. When a proper match or comparison is obtained by controller 17, the drop generator(s) 18 may be enabled.

In this alternate embodiment, the system 100 or inhaler 10 is in a non-operable state when the enabling information has not yet been inputted. As such, the user may not use the system 100 or inhaler 10 at this point. This may be advantageous in that, if someone steals, or in any other unauthorized manner acquires inhaler 10, the enabling information generally will not have been input into the inhaler 10, thus rendering the inhaler substantially useless.

Once the enabling information is stored within the storage device 21, the system 100 or inhaler 10 shifts to an operable locked state. This “locked” state substantially prevents a user from receiving the pharmaceutically active ingredient 12 until an operational condition is received and recognized as substantially matching the enabling information.

The sensing device 19 may recognize and/or receive one or more operational conditions. The electronic circuitry 20 is adapted to compare the previously saved enabling information with the received operational condition(s). A comparison result is generated, and if the comparison result is in an acceptable predetermined range, the drop generator 18 of the system 100 or inhaler 10 is activated, and the system 100 or inhaler 10 shifts to an operable unlocked state. During this “unlocked” state, the user may receive the pharmaceutically active ingredient 12. It is to be understood, however, that after such use, the system 100 or inhaler 10 reverts back to the operable locked state, which may be again unlocked upon recognition and acceptance (i.e. a favorable comparison of operational condition(s) and enabling information) of a subsequent operational condition.

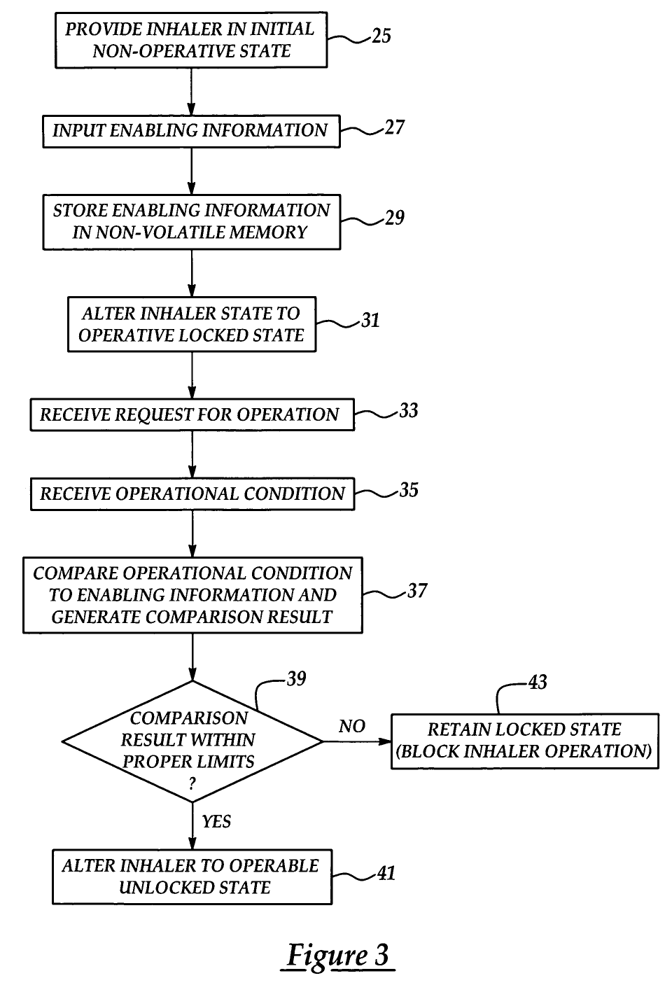

Referring now to FIG. 3, an embodiment of a method using the comparison of enabling information and operational condition(s) is depicted. The inhaler 10 is provided in its initial non-operational state, as shown at reference numeral 25. The selected enabling information is inputted and saved into the NVM storage device 21, as shown at reference numerals 27 and 29. Upon saving the enabling information, the inhaler 10 is switched to the operative locked state, depicted at reference numeral 31. A user then attempts to operate the inhaler 10, whereby the inhaler recognizes a request for such operation, as shown at reference numeral 33. The sensing device 19 recognizes an operational condition (non-limitative examples of which include a key code entered by the user, a date code, biometric information of the current user, and the current fluid capacity of the reservoir 14), as shown at reference numeral 35. The electronic circuitry 20 is capable of comparing the stored enabling information to the received operational condition and generating a comparison result therefrom, as at reference numeral 37. If the comparison result is within proper predetermined limits (generally based on the enabling information), the inhaler 10 is switched to an operable unlocked state which activates or allows activation of the drop generator 18, thereby allowing the user to generate an aerosol of the pharmaceutically active ingredient 12, as shown at reference numeral 41. If the comparison result is outside of the proper predetermined limits, the inhaler 10 remains in the operable locked state, thereby blocking the release of the pharmaceutically active ingredient, as shown at reference numeral 43.

It is to be understood that the previously described storing and comparing capabilities of the various components of the electronic circuitry 20 may also be used in an embodiment of the system 100 or inhaler 10 where the drop generator 18 is deactivated. A non-limitative example of this is shown in FIG. 4. Therefore, referring now to FIG. 4, the sensing device 19 may be a biometric input device that receives a signal carrying first information indicative of, for example, the identity of a person (detected by, for example, a fingerprint recognized by a fingerprint detecting chip) attempting to utilize inhaler 10 or system 100, as shown at reference numeral 45. The controller 17 of the electronic circuitry 20 may receive the activation signal generated by the sensing device 19, as shown at reference numeral 47. The storage device 21 (a non-limitative example of which is a non-volatile memory device) has stored therein second information indicative of an authorized user (e.g. spline representation of the authorized user's fingerprint) of inhaler 10, and the controller 17 receives such information, as shown at reference numeral 49. The controller 17 of the electronic circuitry 20 compares this first information with the second information that is stored on or in the storage device 21, as at reference numeral 51. If the first information is found to not properly compare with the second information, a fault signal is then imparted to the controller 17, as shown at reference numeral 53. In response to the fault signal, the electronic circuitry 20 deactivates the drop generator(s) 18, as shown at reference numeral 55. If however, the first information substantially matches the second information, an aerosol of the pharmaceutically active ingredient 12 is generated, as shown at reference numeral 57.

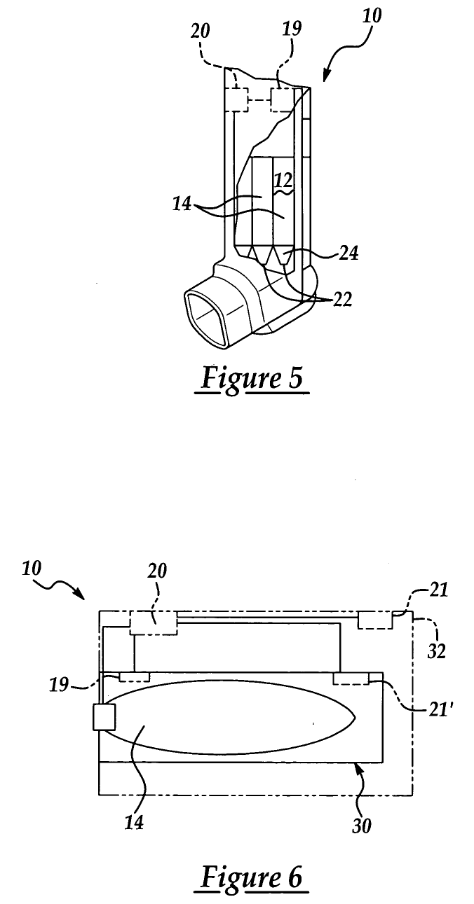

Referring now to FIG. 5, an embodiment of an inhaler 10 is depicted. The inhaler 10 includes a drop generator 18 that releases a particular substance from within the inhaler 10 to, for example, a user's mouth, nose, etc. As such, the inhaler 10 may be a nasal inhaler and/or an oral inhaler (depicted in FIG. 5). Generally, an embodiment of the inhaler 10 uses drop generating technology to form an aerosol of the pharmaceutically active ingredient 12.

In an embodiment, the drop generator 18 includes an orifice 22 associated with a drop ejector 24. The reservoir(s) 14 (having the pharmaceutically active ingredient 12 disposed therein) are selectively fluidly coupled to the drop ejector(s) 24 and orifice(s) 22 (i.e. the drop generator(s) 18). In an embodiment, the drop ejector 24 ejects discrete droplet(s) from the reservoir 14 through the orifice 22 in response to receiving a current or voltage pulse.

An embodiment of the inhaler 10 includes the electronic/control circuitry 20 in electronic communication with, and operatively controlling the drop generator 18. In an embodiment, under normal operation of the inhaler 10, the control circuitry 20 electronically activates the elements of the drop generator 18 to atomize and release the fluid drops to the user. Upon recognition of a predetermined fault condition, however, the electronic control 20 electronically deactivates the elements of the drop generator 18 to prevent the release of fluid drops to the user. In an alternate embodiment, the inhaler 10 is in a locked state until the recognition and acceptance of an operational condition that substantially matches predetermined limits of previously stored enabling information. Upon such recognition and acceptance, the electronic circuitry 20, and in particular the drive circuitry 23, electronically activates the elements of the drop generator 18 to atomize and release the fluid drops to the user.

In a non-limitative example embodiment, the drop ejector 24 of an oral inhaler releases discrete droplet(s) having average diameter(s) ranging between about 1 μm and about 20 μm. For nasal inhalers, generally the discrete droplet(s) have average diameters greater than about 20 μm.

In an embodiment, the inhaler 10 may optionally include an electronic sensing device 19 that is capable of sensing the one or more predetermined fault conditions or the one or more operational conditions. In this embodiment, the sensing device 19 is operatively connected to the inhaler 10 and is in electrical communication with and/or forms a portion of the electronic circuitry 20. Upon sensing a fault condition, the electronic sensing device 19 may signal the controller 17 and the drive circuitry 23 of the electronic circuitry 20, which in turn deactivates the drop generator 18. Upon sensing an operational condition, the electronic sensing device 19 may signal the controller 17 to compare the information, which upon acceptance of the comparison, signals the drive circuitry 23 to activate the drop generator 18. It is to be understood that the electronic sensing device 19 may also be operatively connected to an embodiment of the system 100 as described herein.

FIG. 6 schematically depicts a portion of an alternate embodiment of the inhaler 10. The reservoir 14 (and optionally the sensing device 19) and the storage device 21 are contained within a cartridge 30 that is removable from a housing 32 that makes up the exterior of the inhaler 10. The housing 32 may contain an access door (not shown) for easy insertion and/or removal of the cartridge 30. When the cartridge 30 is disposed within the housing 32, it is to be understood that the electronic circuitry 20 may be configured such that it becomes operatively connected to the sensing device 19 (if present), the storage device 21 (if present), and the drop ejector 16 (not shown).

The storage device 21, shown in FIG. 6, is located in the housing 32. It is to be understood that the storage device 21 may be located in the cartridge 30 rather than in the housing 32. Still further, a first storage device 21 may be located in the housing 32 while a second storage device 21′ may be located in the cartridge 30. In an embodiment having first and second storage devices 21, 21′, the first storage device 21 may store enabling information such as key codes and/or biometric input while the second storage device 21′ may store enabling information such as key codes, date codes, and/or fluid capacity.

Referring now to FIG. 7, an embodiment of the ejector head 16 is depicted. Non-limitative examples of suitable ejector heads 16 include thermal drop ejection mechanisms, piezo drop ejection mechanisms, electrohydrodynamic drop ejection mechanisms, mechanical extrusion drop ejection mechanisms, and combinations thereof.

The ejector head 16 may include one or more drop generators 18, which include the drop ejector 24 in selective fluid communication with the reservoir 14, and at least one orifice 22 through which the droplet(s) is eventually ejected. The elements of the drop generator 18 may be electronically activated to release the fluid drops. It is to be understood that the drop generators 18 may be positioned as a linear or substantially non-linear array, or as an array having any two dimensional shape, as desired.

Drop generator circuitry 20′ may be included in the ejector head 16. Non-limitative examples of drop generator circuitry 20′ include thin film circuitry or thin film devices that define drop ejection elements, such as resistors or piezo-transducers. Still further, the drop generator circuitry 20′ may include drive circuitry such as, for example, transistors, logic circuitry, and input contact pads. In one embodiment, the thin film circuitry includes a resistor configured to receive current pulses and to generate thermally generated bubbles in response. In another embodiment, the thin film device includes a piezo-electrical device configured to receive current pulses and to change dimension in response thereto.

It is to be understood that the drop generator circuitry 20′ of the ejector head 16 may receive electrical signals and in response, may activate, or deactivate, one or more of the array of drop generators 18. Each drop generator 18 is pulse activated, such that it ejects a discrete droplet in response to receiving a current or voltage pulse. Each drop generator 18 may be addressed individually, or groups of drop generators 18 may be addressed substantially simultaneously.

A non-limitative example of the ejector head 16 includes a substrate 28 having a plurality of drop generators 18 established thereon. Any suitable substrate 28 may be selected, and in a non-limitative embodiment, the substrate 28 is one or more of single crystal silicon, polycrystalline silicon, silicon oxide containing dielectric substrates, alumina, sapphire, ceramic, glass, silicon wafers, plastics and/or mixtures thereof.

FIG. 8 is a cross-sectional semi-schematic view taken along line 8-8 of FIG. 7, depicting an enlarged view of an orifice 22 and the drop generator(s) 18.

Embodiments of the system 100, inhaler 10, and methods disclosed herein offer many advantages, including, but not limited to the following. The drop generator(s) 18 is advantageously controlled such that, upon exposure to and/or recognition of certain predetermined fault conditions, the drop generator 18 is deactivated/disabled. Deactivation of the drop generator 18 may advantageously prevent the pharmaceutically active ingredient 12 from being ejected from the inhaler 10 or system 100. Non-limitative examples of when the drop generator 18 may be rendered disabled include expiration of the ingredient 12, unauthorized use of the pharmaceutically active ingredient 12, tampering with the inhaler 10/system 100, user request, etc. It is to be understood that when the drop generator 18 is rendered disabled, the user is advantageously substantially prevented from potentially abusing the pharmaceutically active ingredient 12.

Alternatively, the inhaler 10 may advantageously remain deactivated until recognition and acceptance of certain operational conditions. The locked state advantageously prevents the pharmaceutically active ingredient 12 from being ejected from the inhaler 10 or system 100.

While several embodiments have been described in detail, it will be apparent to those skilled in the art that the disclosed embodiments may be modified. Therefore, the foregoing description is to be considered exemplary rather than limiting.

What is claimed is:

1. An inhaler, comprising:
an ejector head including at least one drop generator;
a reservoir in selective fluid communication with the at least one drop generator, the reservoir adapted to contain a pharmaceutically active ingredient; and
electronic circuitry responsive to at least one predetermined fault condition, the electronic circuitry in electronic communication with, and operatively controlling the at least one drop generator;
wherein in response to the at least one predetermined fault condition, the electronic circuitry deactivates the at least one drop generator.

2. The inhaler as defined in claim 1 wherein the at least one predetermined fault condition includes at least one of tampering, reuse of a single use reservoir, reservoir leaking, pharmaceutically active ingredient expiration, pharmaceutically active ingredient overuse, attempted re-use after inhaler disposal, unauthorized use, loss of reservoir back pressure, loss of electronic circuitry power, user request, and combinations thereof.

3. The inhaler as defined in claim 1 wherein the electronic circuitry substantially permanently deactivates the at least one drop generator.

4. The inhaler as defined in claim 3 wherein, upon recognizing the at least one predetermined fault condition, the electronic circuitry imparts an electrical surge to the at least one drop generator, thereby rendering the at least one drop generator permanently deactivated.

5. The inhaler as defined in claim 1, wherein the at least one drop generator includes a drop ejector and an orifice associated with the drop ejector.

6. The inhaler as defined in claim 1, further comprising an electronic sensing device operatively connected to the inhaler and adapted to sense the at least one predetermined fault condition.

7. The inhaler as defined in claim 6 wherein the sensing device is in communication with the electronic circuitry and is adapted to selectively signal the electronic circuitry.

8. The inhaler as defined in claim 1 wherein the ejector head is at least one of thermal drop ejection mechanisms, piezo drop ejection mechanisms, electrohydrodynamic drop ejection mechanisms, mechanical extrusion drop ejection mechanisms, and combinations thereof.

9. The inhaler as defined in claim 1 wherein the electronic circuitry includes at least one of a controller, a sensing device, drive circuitry, a storage device, and combinations thereof.

10. The inhaler as defined in claim 1 wherein the ejector head includes an array of drop generators.

11. The inhaler as defined in claim 1 wherein the electronic circuitry includes drive circuitry which accomplishes the deactivation of the at least one drop generator.

12. The inhaler as defined in claim 1 wherein the reservoir contains the pharmaceutically active ingredient.

13. A method for making a pharmaceutically active ingredient abuse-prevention device, the method comprising:
providing an ejector head including at least one drop generator;
providing a pharmaceutically active ingredient in selective fluid communication with the at least one drop generator; and
configuring electronic circuitry to selectively operate the at least one drop generator such that, upon recognition of at least one predetermined fault condition, the at least one drop generator is deactivated.

14. The method as defined in claim 13 wherein the at least one predetermined fault condition includes at least one of tampering, reuse of a single use reservoir, reservoir leaking, pharmaceutically active ingredient expiration, pharmaceutically active ingredient overuse, attempted re-use after inhaler disposal, unauthorized use, loss of reservoir back pressure, loss of electronic circuitry power, user request, and combinations thereof.

15. The method as defined in claim 14 wherein the electronic circuitry is configured to sense the at least one predetermined fault condition and is in electronic communication with, and operatively controls the at least one drop generator.

16. The method as defined in claim 15 wherein upon recognition of the at least one predetermined fault condition, the electronic circuitry imparts an electrical surge to the at least one drop generator, thereby rendering the at least one drop generator permanently deactivated.

17. The method as defined in claim 13 wherein the ejector head is at least one of thermal drop ejection mechanisms, piezo drop ejection mechanisms, electrohydrodynamic drop ejection mechanisms, mechanical extrusion drop ejection mechanisms, and combinations thereof.

18. The method as defined in claim 13 wherein the electronic circuitry includes at least one of a controller, a sensing device, drive circuitry, a storage device, and combinations thereof.

19. The method as defined in claim 18 wherein the drive circuitry deactivates the at least one drop generator.

20. The method as defined in claim 13 wherein the ejector head includes an array of drop generators.

21. The method as defined in claim 13, further comprising:
disposing the pharmaceutically active ingredient in a reservoir; and
operatively and fluidly connecting the reservoir to the at least one drop generator.

22. An inhaler made by the method as defined in claim 13.

23. A system for preventing abuse of a pharmaceutically active ingredient, the system comprising:
an ejector head including an array of drop generators;
a reservoir in selective fluid communication with the array of drop generators, the reservoir adapted to contain the pharmaceutically active ingredient; and
electronic circuitry responsive to at least one predetermined fault condition, the electronic circuitry in electronic communication with, and operatively controlling the array of drop generators;
wherein in response to the at least one predetermined fault condition, the electronic circuitry deactivates the array of drop generators.

24. The system as defined in claim 23 wherein the at least one predetermined fault condition includes at least one of tampering, reuse of a single use reservoir, reservoir leaking, pharmaceutically active ingredient expiration, pharmaceutically active ingredient overuse, attempted re-use after inhaler disposal, unauthorized use, loss of reservoir back pressure, loss of electronic circuitry power, user request, and combinations thereof.

25. The system as defined in claim 23 wherein the electronic circuitry includes drive circuitry adapted to substantially permanently deactivate the array of drop generators.

26. The system as defined in claim 23 wherein, upon recognizing the at least one predetermined fault condition, the electronic circuitry imparts an electrical surge to the array of drop generators, thereby rendering the array of drop generators substantially permanently deactivated.

27. The system as defined in claim 23, wherein each drop generator in the array includes a drop ejector and an orifice associated with the drop ejector.

28. The system as defined in claim 23, further comprising an electronic sensing device operatively connected to the system and adapted to sense the at least one predetermined fault condition.

29. The system as defined in claim 28 wherein the sensing device is in communication with the electronic circuitry and is configured to selectively signal the electronic circuitry.

30. The system as defined in claim 23 wherein the ejector head is at least one of thermal drop ejection mechanisms, piezo drop ejection mechanisms, electrohydrodynamic drop ejection mechanisms, mechanical extrusion drop ejection mechanisms, and combinations thereof.

31. The system as defined in claim 23 wherein the electronic circuitry includes at least one of a controller, a sensing device, drive circuitry, a storage device, and combinations thereof.

32. The system as defined in claim 23 wherein the reservoir contains the pharmaceutically active ingredient.

33. An inhaler, comprising:
an ejector head including at least one drop generator;
a reservoir in selective fluid communication with the at least one drop generator, the reservoir adapted to contain a pharmaceutically active ingredient;
electronic circuitry responsive to at least one operational condition, the electronic circuitry in electronic communication with, and operatively controlling the at least one drop generator; and
a storage device in electronic communication with the electronic circuitry, the storage device adapted to store enabling information;
wherein in response to the at least one operational condition, the electronic circuitry compares the at least one operational condition to stored enabling information and activates the at least one drop generator if the at least one operational condition and the enabling information substantially match.

34. The inhaler as defined in claim 33 wherein the enabling information includes at least one of a key code, a date code, reservoir fluid capacity, biometric input, and combinations thereof.

35. The inhaler as defined in claim 33 wherein the at least one drop generator includes a drop ejector and an orifice associated with the drop ejector.

36. The inhaler as defined in claim 33 wherein the storage device is a non-volatile memory device.

37. The inhaler as defined in claim 33 wherein prior to activating the at least one drop generator, the inhaler is in at least one of a non-operable state and an operable locked state.

38. The inhaler as defined in claim 33, further comprising an electronic sensing device operatively connected to the inhaler and adapted to sense the at least one operational condition.

39. The inhaler as defined in claim 38 wherein the sensing device is in communication with the electronic circuitry and is adapted to selectively signal the electronic circuitry.

40. The inhaler as defined in claim 33 wherein the ejector head is at least one of thermal drop ejection mechanisms, piezo drop ejection mechanisms, electrohydrodynamic drop ejection mechanisms, mechanical extrusion drop ejection mechanisms, and combinations thereof.

41. The inhaler as defined in claim 33 wherein the electronic circuitry includes drive circuitry adapted to activate the at least one drop generator.

42. The inhaler as defined in claim 33 wherein the ejector head includes an array of drop generators.

43. The inhaler as defined in claim 33, further comprising a housing having the reservoir operatively disposed therein.

44. The inhaler as defined in claim 43, further comprising a cartridge removably disposed within the housing, the cartridge having disposed therein the reservoir and the storage device.

45. The inhaler as defined in claim 44 wherein the storage device is a non-volatile memory device and wherein the enabling information is at least one of a key code, a date code, and reservoir fluid capacity.

46. The inhaler as defined in claim 43, further comprising a cartridge removably disposed within the housing, the cartridge having disposed therein the reservoir and a second storage device storing second enabling information.

47. The inhaler as defined in claim 46 wherein the enabling information is at least one of a key code and biometric input and wherein the second enabling information is at least one of a key code, a date code, and reservoir fluid capacity.

48. The inhaler as defined in claim 33 wherein the reservoir contains the pharmaceutically active ingredient.

49. A method for making a pharmaceutically active ingredient abuse-prevention device, the method comprising:
providing an ejector head including at least one drop generator;
providing a pharmaceutically active ingredient in selective fluid communication with the at least one drop generator;
configuring electronic circuitry to selectively operate the at least one drop generator and to be responsive to at least one operational condition; and
operatively connecting a storage device to the electronic circuitry such that, upon recognition of at least one operational condition, the electronic circuitry compares the at least one operational condition to enabling information stored in the storage device and enables activation of the at least one drop generator if the at least one operational condition and the enabling information substantially match.

50. The method as defined in claim 49 wherein the ejector head is at least one of thermal drop ejection mechanisms, piezo drop ejection mechanisms, electrohydrodynamic drop ejection mechanisms, mechanical extrusion drop ejection mechanisms, and combinations thereof.

51. The method as defined in claim 49 wherein the electronic circuitry includes at least one of a controller, a sensing device, drive circuitry, and combinations thereof.

52. The method as defined in claim 51 wherein the drive circuitry accomplishes the activation of the at least one drop generator.

53. The method as defined in claim 49 wherein the enabling information includes at least one of a key code, a date code, reservoir fluid capacity, biometric input, and combinations thereof.

54. The method as defined in claim 49 wherein the at least one drop generator includes a drop ejector and an orifice associated with the drop ejector.

55. The method as defined in claim 49 wherein the storage device is a non-volatile memory device.

56. The method as defined in claim 49 wherein the electronic circuitry includes an electronic sensing device operatively connected to the device and adapted to sense the at least one operational condition.

57. The method as defined in claim 56 wherein the sensing device is in communication with the electronic circuitry and is adapted to selectively signal the electronic circuitry.

58. The method as defined in claim 49 wherein the ejector head includes an array of drop generators.

59. The method as defined in claim 49, further comprising:
disposing the pharmaceutically active ingredient in a reservoir; and
operatively and fluidly connecting the reservoir to the at least one drop generator.

60. An inhaler made by the method as defined in claim 49.

61. An inhalation device, comprising:
an ejector head including at least one drop generator;
a reservoir in selective fluid communication with the at least one drop generator, the reservoir adapted to contain a pharmaceutically active ingredient; and
electronic circuitry responsive to one of a predetermined fault condition and an operational condition, the electronic circuitry in electronic communication with, and operatively controlling the at least one drop generator;
wherein in response to the predetermined fault condition, the electronic circuitry deactivates the at least one drop generator, and wherein in response to the operational condition, the electronic circuitry activates the at least one drop generator.

62. The inhalation device as defined in claim 61 wherein the predetermined fault condition includes at least one of tampering, reuse of a single use reservoir, reservoir leaking, pharmaceutically active ingredient expiration, pharmaceutically active ingredient overuse, attempted re-use after inhaler disposal, unauthorized use, loss of reservoir back pressure, loss of electronic circuitry power, user request, and combinations thereof.

63. The inhalation device as defined in claim 61, further comprising a storage device operatively connected to the electronic circuitry and adapted to store enabling information; wherein the electronic circuitry compares the operational condition to enabling information stored in the storage device and enables activation of the at least one drop generator if the operational condition and the enabling information substantially match.

64. The inhalation device as defined in claim 63 wherein the storage device is a non-volatile memory device.

65. The inhalation device as defined in claim 63 wherein the enabling information includes at least one of a key code, a date code, reservoir fluid capacity, biometric input, and combinations thereof.

66. The inhalation device as defined in claim 61 wherein the electronic circuitry includes at least one of a controller, a sensing device, drive circuitry, a storage device, and combinations thereof.

67. The inhalation device as defined in claim 61 wherein the ejector head is at least one of thermal drop ejection mechanisms, piezo drop ejection mechanisms, electrohydrodynamic drop ejection mechanisms, mechanical extrusion drop ejection mechanisms, and combinations thereof.

68. The inhalation device as defined in claim 61 wherein the ejector head includes an array of drop generators.

69. The inhalation device as defined in claim 61 wherein the reservoir contains the pharmaceutically active ingredient.
